(19) United States
(12) Patent Application Publication
(10) Pub. No.: US 2024/0025819 A1
(43) Pub. Date: Jan. 25, 2024

(54) METHOD AND SYSTEM FOR PRODUCING ONE OR MORE OLEFINS

(71) Applicant: LINDE GMBH, Pullach (DE)

(72) Inventors: Andreas MEISWINKEL, Rimsting (DE); Hans-Jörg ZANDER, München (DE)

(21) Appl. No.: 17/793,306

(22) PCT Filed: Jan. 14, 2021
(86) PCT No.: PCT/EP2021/050686

§ 371 Date: Jul. 15, 2022

(30) Foreign Application Priority Data
Jan. 17, 2020 (DE) 10 2020 101 054.0

(51) Int. Cl. C07C 1/12; B01J 19/24

(52) U.S. Cl. CPC C07C 1/12; B01J 19/245; B01J 2219/0004

(57) ABSTRACT

Disclosed is a method for producing olefins having a carbon number of two to eight, in which carbon dioxide and hydrogen are fed to a hydrogenation step, and, wherein portions of carbon dioxide and hydrogen are then converted with one another on a bifunctional catalyst via an oxygenate as an intermediate into the one or more olefins. It is provided that the hydrogen fed to the hydrogenation step is provided by a reforming step, in which methane and water are converted into hydrogen, carbon monoxide, and carbon dioxide, and that the carbon dioxide that is fed to the hydrogenation step is provided in part using the reforming step and in part independently of the reforming step. Providing the portions of hydrogen and carbon dioxide comprises providing, using the reforming step, a gas mixture containing hydrogen, carbon monoxide and carbon dioxide and feeding at least a portion of the gas mixture to the hydrogenation step without separating hydrogen, carbon monoxide, and carbon dioxide.

The project that has led to the present patent application was promoted within the framework of financial aid agreement no. 837733 of the European Union's Horizon 2020 Research and Innovations program.

### BACKGROUND

### 1. Field of the Invention

The present invention relates to a method for producing one or more olefins, and to a corresponding system.

### 2. Description of the Related Art

The production of propylene (propene) is described in the specialist literature, for example in the article “Propylene” in Ullmann's Encyclopedia of Industrial Chemistry, ed. 2012. Propylene is conventionally produced by steam-cracking hydrocarbon feeds and by conversion processes in the course of refinery processes. In the latter processes, propylene is not necessarily formed in the desired quantity and only as one of several components in a mixture with further compounds. Other methods for producing propylene are likewise known and are described further below in connection with the present invention. They are however not satisfactory in all cases, for example in terms of efficiency and yield.

WO 2008/131898 A1 discloses a method for producing a synthesis gas mixture containing hydrogen, carbon monoxide, and carbon dioxide, wherein the method comprises a step in which a gaseous feed mixture containing carbon dioxide and hydrogen is brought into contact with a catalyst, wherein the catalyst substantially consists of manganese oxide and an oxide of at least one element selected from the group of chromium, nickel, lanthanum, cerium, tungsten, and platinum. The method is intended to enable the hydrogenation of carbon dioxide to form carbon monoxide with high selectivity and good catalyst stability over time and under different processing conditions. The method can be applied separately but also integrated into other processes, both upstream and downstream, such as methane reforming or other synthesis methods for producing products such as alkanes, aldehydes, or alcohols.

An increasing demand for propylene (“propylene gap”), which requires the provision of corresponding methods, is predicted for the future. At the same time, it is necessary to reduce or even prevent carbon dioxide emissions. As a potential feedstock, on the other hand, large amounts of methane are available, which are currently only fed to a material utilization in a very limited manner and are predominantly burned.

### SUMMARY

The object of disclosed embodiments is to provide an improved method, in particular in view of these aspects, for producing propylene but also for producing other comparatively short-chain olefins.

This object is achieved by a method for producing one or more olefins and by a corresponding system with the respective features of the independent claims. Embodiments are the subject-matter of the dependent claims and of the following description.

Overall, the embodiments propose a reaction system for converting carbon dioxide with hydrogen into hydrocarbons in a hydrogenation step, wherein the hydrogenation step is combined with a reforming step. In the reforming step, methane (and, where applicable, further hydrocarbons; methane, however, is the preferred main component since a maximum hydrogen yield is achieved with methane) is converted with steam into hydrogen and carbon monoxide. A shift reaction, which is frequently downstream according to the current state of the art, can be omitted in embodiments of the invention since the catalyst used in the hydrogenation step also converts carbon monoxide into hydrocarbons.

Within the framework of the disclosed embodiments, the reforming step is in particular a hydrogen source but, with carbon monoxide and/or carbon dioxide, also provides further starting material, which can be converted into hydrocarbons in the hydrogenation step. The hydrogen, as also explained below, accumulates here in stoichiometric excess and can thus be used in particular for converting further carbon dioxide from an external source. In this way, a saving or a meaningful material utilization of carbon dioxide from corresponding sources can be achieved by means of the embodiment.

The disclosed embodiments overall provide a particularly efficient conversion of carbon dioxide with hydrogen into valuable products. Within the framework of the disclosed embodiments, both carbon dioxide from a source in which it accumulates as an unwanted product and the carbon dioxide produced in certain quantities (but advantageously here in a smaller quantity than carbon monoxide) as a by-product in the reforming step can be converted into valuable products, i.e., in the ideal case, carbon dioxide emissions can be completely prevented as a result of the present invention. In summary, within the framework of the present invention, both carbon dioxide and methane can be introduced into material use.

As also explained below, by using the reforming step, the disclosed embodiments also allow a simple return of by-products (in particular paraffins) and of unconverted reactants, such as hydrogen, carbon monoxide, carbon dioxide, methane, and/or of hydrocarbons undesired as product, to the reforming and/or hydrogenation step in a selective and expedient manner. In this way, undesired side streams can be avoided as products and can be materially utilized within the method.

A particular advantage, which results from the material use of all components from the reforming step, is that they do not have to be separated from one another or only have to be partly separated from one another before they are fed to the hydrogenation step. In other words, the disclosed embodiments enable minimization of the purification and conditioning of the hydrogen used in the hydrogenation step, and thus the minimization of production and operating costs. In particular, components that should not be introduced into the hydrogenation step because they would interfere there, do not accumulate in the reforming step.

In order to achieve the aforementioned advantages and to implement the method steps already mentioned in the terminology of the claims, the disclosed embodiments provide a method for producing one or more olefins with a carbon number of two to eight, in particular of three to eight or three to six, in which method carbon dioxide and hydrogen are fed to a hydrogenation step, wherein at least a portion of the carbon dioxide and at least a portion of the hydrogen that are fed to the hydrogenation step are converted with one another in the hydrogenation step on a bifunctional catalyst via an oxygenate, in particular an alcohol or an ether, further in particular methanol or dimethyl ether, as an intermediate into the one or more olefins.

The term “oxygenate” is to be understood in accordance with a common definition, which also applies in the present case, as meaning compounds that have at least one alkyl group covalently bonded to an oxygen atom. The at least one alkyl group may have up to five, up to four, or up to three carbon atoms. In particular, the oxygenates that are of interest within the framework of the present invention have alkyl groups with one or two carbon atoms, are in particular methyl groups. In particular, they are monovalent alcohols and dialkyl ethers, such as methanol, ethanol, tert-butanol (TBA), and dimethyl ether (DME), or corresponding mixtures. Examples of further oxygenates are methyl tert-butyl ether (MTBE), tert-amyl methyl ether (TAME), tert-amyl ethyl ether (TAEE), ethyl tert-butyl ether (ETBE), and diisopropyl ether (DIPE). The invention is also basically suitable for use with other oxygenates but is predominantly described with reference to methanol and dimethyl ether.

Within the framework of the disclosed embodiments, in particular short-chain alcohols and ethers are thus relevant as oxygenates, in particular monovalent or divalent alcohols having a carbon number of one, two, or three and dialkyl ethers having a carbon number of two, three, or four.

According to the embodiments, the hydrogen that is fed to the hydrogenation step is provided at least in part by means of a reforming step in which methane and water are converted into carbon dioxide, carbon monoxide, and hydrogen, and the carbon dioxide that is fed to the hydrogenation step is provided in part using the reforming step and in part independently of the reforming step.

According to the embodiments, providing at least a portion of the hydrogen, a portion of the carbon monoxide, and a portion of the carbon dioxide by means of the reforming step for the hydrogenation step comprises in all cases providing, using the reforming step, a gas mixture containing hydrogen, carbon monoxide, and carbon dioxide and feeding at least a portion of the gas mixture to the hydrogenation step without separating hydrogen, carbon monoxide, and carbon dioxide.

### BRIEF DESCRIPTION OF THE DRAWINGS

FIG. 1 shows an embodiment of a plant according to embodiments in the form of a schematic block diagram.

### DETAILED DESCRIPTION

In what follows, backgrounds of the hydrogenation provided within the framework of the present invention are first explained in relation to further prior art for producing olefins, in particular propylene, before further features and advantages of the invention are addressed.

The disclosed embodiments can be used in particular in connection with efforts to reduce the emission of carbon dioxide and to materially use carbon dioxide. General review articles for the use and conversion of carbon dioxide are indicated by [0a] to [0f] in the enclosed literature overview. Reference is made here and in the following to the corresponding literature.

As mentioned, for the production of propylene, but also in particular of ethylene, as a conventional main target component from hydrocarbons or corresponding mixtures (e.g. ethane, propane, liquefied natural gas, and naphtha) is known. In particular, fluid catalytic cracking is also used for the production of propylene. In this context, reference is made, for example, to citation [1a], which provides a current overview of new routes to olefins.

In particular, alternative technologies exist for the production (a) of ethylene from ethane, in particular by oxidative dehydrogenation of ethane, in which, however, acetic acid has also always been produced in certain quantities so far as a coupling product, (b) of ethylene by oxidative coupling of methane, which coupling is however currently still in a comparatively earlier development stage and is limited to low conversions or yields, (c) of propylene from propane, in particular by propane dehydrogenation, which is a commercially available and established method, (d) of propylene from ethylene by olefin metathesis, in which 2-butene is always also required as input but which is likewise commercially available and established, (e) of olefins and in particular propylene by so-called methanol-to-olefin or methanol-to-propylene methods, in which syngas (e.g., produced from methane but also from coal, naphtha, etc.) is first converted into methanol and/or dimethyl ether and the latter are then converted into olefins or propylene after isolation by a further conversion in a separate method step, (f) of olefins by modified Fischer-Tropsch methods, which are optimized in such a way that a maximum yield of lightweight olefins (Fischer-Tropsch-to-olefin) results, and (g) of olefins, in the case of which corresponding Fischer-Tropsch methods are combined with a reverse water gas shift, and which therefore also incorporate carbon dioxide.

An overview of catalysts for Fischer-Tropsch-to-olefin methods can be found, for example, in citation [2x], where iron-based and cobalt-based catalysts are in particular compared. The latter are also described in WO 2012/084160, for example. Citation [2y] likewise offers an overview of Fischer-Tropsch-to-olefin methods.

The Fischer-Tropsch method with a reverse water gas shift is, for example, rather generally described in citations [2a] to [2c] and in a more detailed manner in citations [2r] and [2s].

For producing synthesis gas (syngas), which is to be understood here to mean a gas mixture with the components carbon monoxide and hydrogen, optionally containing carbon dioxide in certain quantities, steam reforming is known and described extensively in the technical literature. The existing variants of steam reforming are (a) so-called dry reforming and (b) steam reforming with downstream water gas shift for adjusting the ratio of hydrogen and carbon monoxide. However, neither variant is necessarily important for the disclosed embodiments. Steam reforming is described in general in citations [3a] to [3e].

Oxygenates, in particular methanol and/or DME, can be produced from synthesis gas which, as explained above, can typically be produced by steam reforming. Methanol synthesis is described, for example, in citations [3f] and [3g]. The further conversion of oxygenates according to the above definition, in particular of methanol and/or dimethyl ether, into olefins is discussed, for example, in citation [3h]. Furthermore, citations [3i] and [3j], for example, show approaches for combining methanol synthesis from synthesis gas and a methanol-to-olefin method in one step. These approaches are based essentially on a combination of Zr—Zn oxides for methanol synthesis and H-SAPO-34 for the methanol-to-olefin method.

As a further approach of the last point, there are furthermore developments for the hydrogenation of carbon dioxide via bifunctional catalysts. The disclosed embodiments start at this point. This approach via an oxygenate such as methanol and/or dimethyl ether as intermediate can be considered as a combination of the reactions or reaction steps 1 and 2 indicated below:
1) Reaction of carbon dioxide with hydrogen to form the oxygenate as intermediate
2) Consecutive reaction of the intermediate to form hydrocarbons

Step 2 is in particular comparable to the known methanol-to-olefin or methanol-to-propylene methods in which, however, the corresponding intermediate is isolated as mentioned.

Typically, bifunctional catalysts are used which typically combine methanol catalysts with acidic zeolite structures which catalyze the consecutive reaction. The overall reaction takes place as a single-stage reaction, wherein a “single-stage” reaction is to denote a reaction in which the conversion takes place in the two steps on a catalyst or in a catalyst bed but in any case in a reactor and without intermediate separation of the oxygenate.

For example, the following individual reactions result for propylene as target product:

CO₂+3H₂ [symbol] CH₃OH+H₂O ΔH=−48 kJ/mol

3CH3OH [symbol] C₃H₆+3H₂O ΔH=−104 kJ/mol

As a secondary reaction, “overhydrogenation” to form propane may occur:

3CH₃OH+H₂→C₃H₈+3 H₂O ΔH=−227 kJ/mol

For further details, reference is made in particular to citations [4a] to [4j]. A more general overview of carbon dioxide hydrogenation is found, for example, in citation [4k]. Variants of corresponding methods comprise the use of two-stage reaction systems, as described, for example, in citations [4r] and [4s]. However, on the basis of the thermodynamics the latter should be regarded as disadvantageous. Frequently, various olefins, such as ethylene and propylene, as well as different quantities of paraffins (ethane, propane), and also heavier hydrocarbons are formed in corresponding methods. In such methods aromatics can likewise form as by-products.

As mentioned, the disclosed embodiments present invention relates to methods for producing hydrocarbons from carbon dioxide. More particularly embodiments relate to the production of paraffins and particularly preferably of olefins. Paraffins and olefins with the aforementioned chain lengths, particularly preferably propylene, can in particular be obtained. The following embodiments are accordingly partially strongly directed at obtaining propylene but are not limited thereto. As already mentioned, aromatics may also form as by-products.

The disclosed embodiments contribute to covering the mentioned increasing demand for propylene (“propylene gap”) by providing a corresponding selective method in one embodiment. At the same time, the present invention contributes to reducing the carbon dioxide footprint and to significantly reducing or preventing carbon dioxide emissions. Within the framework of the disclosed embodiments, carbon dioxide is converted into a valuable product. As potential further feedstock, the disclosed embodiments use methane, which is available in large quantities but is currently generally fed to material utilization only to a very limited extent.

In contrast to other methods for the selective production of particular olefins, as have been explained above, no or at least no significant quantities of carbon dioxide as occur in alternative methods, either from firing or energy supply in endothermic processes or else as a by-product in oxidative processes, are released within the framework of the present invention. In these methods and processes, carbon monoxide and hydrogen, which have hitherto likewise been fed to material utilization only to a limited extent and are frequently used, for example, for firing endothermic processes, are often also further components that have so far been considered to be undesirable by-products. Within the framework of the disclosed embodiments, they can be directly fed to material conversion.

Current directions of research aim, as explained above, at the conversion of carbon dioxide with hydrogen (where applicable also in the presence of carbon monoxide) into hydrocarbons. The focus here is in particular on paraffins and olefins having two to four carbon atoms. The disclosed embodiments provide an important contribution in this connection.

In general, the hydrogenation of carbon dioxide takes place according to the following equation, where n indicates the chain length of the formed hydrocarbon:

nCO₂+(3n+o)H₂→CₙH₂ₙ₊ₒ+2nH₂O

When o=0, an olefin product is obtained; when o=2, a paraffin product is obtained.

By way of example, the reaction equation for propylene synthesis (n=3 and o=0) is:

3CO₂+9H₂→C₃H₆+6H₂O

In the following, the conversion, just described via a formula, of carbon dioxide with hydrogen in a reactor is referred to in simplified form as “hydrogenation” and the reactor used is referred to as “hydrogenation reactor.”

However, a prerequisite for such methods is that either sufficient hydrogen is already available (e.g., from renewable sources, such as electrolysis, or from existing material streams, where applicable after separation, purification, and/or conditioning). However, this is the case in the fewest cases since often only insufficient quantities of hydrogen are available. It is therefore necessary to provide corresponding (additional) quantities of hydrogen as needed. This takes place in the reforming step within the framework of the disclosed embodiments.

However, technologies, such as reforming or partial oxidation, always also yield carbon monoxide and portions of carbon dioxide as product in addition to hydrogen. Although a shift reaction can in principle increase the proportion of hydrogen, as a result of which additional carbon dioxide is however formed again. This is not expedient and thus in reality only means a loss of efficiency since the hydrogen formed in the shift reaction causes as much carbon dioxide as co-product as then has to be converted again in the hydrogenation reactor.

In particular, a partial oxidation, as an oxidative process, always provides a high proportion of carbon oxides for method-related reasons, thus in particular serves to provide carbon monoxide-rich synthesis gas and thus does not constitute an advantageous hydrogen source here.

In alternative technologies for hydrogen production, electrolysis nowadays has already achieved a high degree of technical maturity and basically provides very pure hydrogen. However, electrolysis still constitutes a relatively expensive type of hydrogen production and can, in particular, be scaled up only to a limited extent. Since this is a “numbering-up” rather than a real “scaling-up,” no economy-of-scale effects in particular or, at best, a linear relationship between system capacity and system costs result. In addition, there is a dependence on the current used, which in the case of conventional technologies, in turn, causes carbon dioxide emissions or, in the case of so-called renewable sources, such as wind and solar electricity, is available only partially or in insufficient or only highly fluctuating quantities.

In contrast, methane as a constituent of natural gas is available in large quantities and is currently in most cases only used for thermal or energetic purposes, while material use takes place only to a very limited extent (e.g., in particular, via syngas-based processes, such as in the aforementioned methanol synthesis).

Both carbon dioxide and, in particular, methane have a very high greenhouse potential and the corresponding emissions accordingly contribute significantly to global warming.

As a result of the measures used in the disclosed embodiments present invention, namely the provision of hydrogen by the reforming in particular of methane, advantageously of natural gas, a hydrogen source is provided which can be integrated easily and economically into a method for converting carbon dioxide into hydrocarbons, which method is suitable in particular for target capacities on an industrial scale (for example, more than 100 kilotonnes per year of hydrocarbon product).

In method embodiments, the methane is thus advantageously provided at least in part using natural gas.

According to the object of the disclosed embodiments, a reaction system for converting carbon dioxide with hydrogen into hydrocarbons is here thus combined with a reformer. The reformer converts methane (and, where applicable, further hydrocarbons, but methane is the preferred main component since a maximum hydrogen yield is achieved here) with steam into hydrogen and carbon monoxide. A downstream shift reaction, as often carried out in established methods, can be omitted in this combination since the catalyst in the hydrogenation reactor also converts carbon monoxide into higher hydrocarbons. In other words, a gas mixture is formed in the reforming step and at least a portion of the gas mixture formed in the reforming step is fed to the hydrogenation step in a material composition that is unchanged, in particular except for the water content.

In the method according to embodiments, carbon monoxide is thus formed in the reforming step and is advantageously fed to the hydrogenation step. In the syngas from the reforming step, significantly less carbon dioxide is advantageously present than carbon monoxide, the molar ratio of carbon monoxide to carbon dioxide is in particular not less than 1:1, in particular not less than 2:1, and in particular not less than 4:1.

In order to also enable a quantity of hydrogen sufficient for hydrogenation in addition to added carbon dioxide, a ratio of 3:1 is ideally required. This value does not relate to the syngas from the reforming step but to the desired value at the entry into the hydrogenation step.

The gas mixture fed to the hydrogenation step also advantageously contains more than or at least the same quantity of carbon dioxide as carbon monoxide, for example in contrast to known methods as disclosed in US 2007/244000 A1. This results, in particular, from the mentioned addition of carbon dioxide from an external source to the syngas. The molar ratio of carbon monoxide to carbon dioxide is advantageously not more than 1:1 here.

An indication of the ratio of the quantities of carbon dioxide and methane as well as water that are converted in the overall process can be found further below in exemplary embodiments I and II. A greater molar quantity of methane and water than of carbon dioxide is required for method-related reasons. In general, it can be advantageous within the framework of the disclosed embodiments if the molar ratio between the carbon dioxide and the hydrogen that are fed to the hydrogenation step is 1:6 to 1:1, in particular 1:4 to 1:2, and if the molar ratio between the methane and the water that are fed to the reforming step is 1:8 to 4:1, in particular 1:4 to 2:1.

Within the framework of the disclosed embodiments, the carbon dioxide provided using the reforming step is fed to the hydrogenation step in a first gas stream, and the carbon dioxide, which is provided independently of the reforming step, is fed to the hydrogenation step in a second gas stream, wherein a quantity ratio between the first gas stream and the second gas stream is advantageously selected such that the components provided with the first gas stream and the second gas stream (i.e., in particular carbon dioxide, carbon monoxide, and hydrogen) correspond to a stoichiometric demand in the hydrogenation step.

The synthesis gas consisting of carbon monoxide and hydrogen from the reforming step (i.e., the first gas stream) can be used here particularly advantageously as a reference value, because a possible formation of CO₂ in the reforming step can be left out of consideration in this way. In the case of propylene synthesis, a ratio between the first gas stream and the second gas stream (the latter can also consist substantially of carbon dioxide) is advantageously adjusted to a range of 6:1 to 2:1, in particular 4:1 to 2.5:1. An ideal ratio is 3:1. This ratio also applies to higher olefins. With regard to derivation, reference is made to Example II below. For olefins, this therefore generally results in a ratio of 3n/n, i.e., likewise 3:1.

Within the framework of the disclosed embodiments, after the reforming step, the cleaning and separation outlay is minimized and the components carbon monoxide and carbon dioxide can in particular be transferred directly into the hydrogenation reactor after a temperature adjustment of this process stream. Although a prior at least partial water separation after the corresponding heat exchanger is also an option, water forming can also be transferred at least partially as steam into the hydrogenation reactor, where it first has a thermodynamically disadvantageous effect on the first reaction step of oxygenate formation but does not considerably interfere in the overall balance due to the coupling of the oxygenate synthesis and the subsequent further conversion into hydrocarbons. In comparison thereto, in the case of an oxygenate synthesis (in particular methanol and/or DME synthesis) operated in isolation, as complete as possible a removal of water from the synthesis gas is advantageous or required. A particular additional advantage within the meaning of the disclosed embodiments thus results from the combination of reforming with the hydrogenation reactor.

Following the hydrogenation reactor and in particular after cooling in a corresponding heat exchanger, the water is then condensed and separated out. The remaining process gas stream can, in particular, be fed to a carbon dioxide removal, for example by means of an amine scrubbing, in order to separate out unconverted carbon dioxide and return it into the hydrogenation reactor again. Optionally, an alkaline scrubbing for fine cleaning and a dryer are connected downstream according to the requirements of downstream process units in order to prevent icing or freezing of carbon dioxide in cryogenic system parts, for example.

Subsequently, in the case of the production of propylene, the process stream can be fed, in particular, to a separation of hydrocarbons with three and, where applicable, more carbon atoms from lighter components. The bottom stream of this separation can be conducted, if needed, into an optional splitter, where, on the one hand, on-spec (e.g., “polymer-grade”) propylene can be obtained as top product, and propane and, where applicable, higher hydrocarbons can be obtained as bottom product (e.g., for use as fuel or motor fuel).

The lighter components mentioned comprise in particular methane, hydrocarbons having two carbon atoms (ethane, ethylene), and residues of carbon monoxide as well as, where applicable, hydrogen. If considerable quantities of ethylene are present in this stream, they can be separated out as an additional valuable product by means of suitable technologies known to the person skilled in the art (by distillation, adsorption, absorption, by means of membrane-based methods, etc.). The remaining stream is then returned again as input into the reforming step.

More generally speaking, and again in the terminology of the claims, the one olefin formed in the method according to embodiments preferably is propylene or the plurality of olefins comprises propylene, in particular in a molar fraction of more than 50%. In particular, at least a portion of the carbon dioxide that is fed to the hydrogenation step and/or at least a portion of the hydrogen that is fed to the hydrogenation step is not converted in the hydrogenation step. In this way, the one or more olefins can be removed from the hydrogenation step as part of a product mixture which, in addition to the one or more olefins, comprises carbon dioxide and/or hydrogen and/or one or more paraffins as further components, wherein at least a portion of the further components is separated out from the product mixture and is returned in each case at least in part into the hydrogenation step and/or the reforming step. In particular, carbon dioxide and compounds boiling more easily than propylene can successively be separated out here, and these components can be returned in the manner mentioned.

As an embodiment of the embodiments, it is also possible to form a material stream that is conducted into the hydrogenation reactor but also contains further components, such as carbon monoxide and/or hydrogen, in addition to carbon dioxide. This is often the case, for example, with residual refinery gases or gases from the steel industry. In general, however, other sources, for example gas mixtures from the ammonia synthesis or from the cement industry, can also be used. The molar quantity of methane and water that must be conducted via the reforming step is thereby in particular reduced.

In a further embodiment, a reactor used in the reforming step can be electrically heated. As a particular advantage, this results in the avoidance of carbon dioxide emissions from the firing, as a result of which ideally carbon dioxide emissions from the overall process are completely avoided.

Within the framework of the disclosed embodiments, preferred conditions of the individual steps comprise a pressure of 10 to 100 bar, in particular of 12 to 50 or 15 to 30 bar in the reforming step or a corresponding reactor, and a pressure of 1 to 100 bar, in particular of 12 to 50 or of 15 to 30 bar, and a temperature of 100 to 520° C., in particular of 150 to 450° C. or 200 to 400° C., in the hydrogenation step or in a corresponding reactor. Without restriction, known catalysts, as indicated, for example, in the literature cited above, generally come into consideration for the reaction system.

In the method according to embodiments, the reforming step is preferably carried out at a higher pressure level than the hydrogenation step so that the components formed in the reforming step and fed to the hydrogenation step can be transferred into the hydrogenation step without compression.

The disclosed embodiments also extend to a system for producing one or more olefins having a carbon number as explained above, wherein the system has a hydrogenation reactor which is configured to subject carbon dioxide and hydrogen to a hydrogenation step in which at least a portion of the carbon dioxide and at least a portion of the hydrogen that are subjected to the hydrogenation step are converted with one another on a bifunctional catalyst via an oxygenate as an intermediate into the one or more olefins. According to embodiments, a reforming reactor is provided for providing the hydrogen that is fed to the hydrogenation step, which reforming reactor is configured to carry out a reforming step in which methane and water are converted into hydrogen, carbon monoxide, and carbon dioxide, and the system is further configured according to embodiments, to provide the carbon dioxide that is fed to the hydrogenation step, in part using the reforming step and in part independently of the reforming step. Providing at least a portion of the hydrogen and a portion of the carbon dioxide by means of the reforming step comprises providing, using the reforming step, a gas mixture containing hydrogen, carbon monoxide, and carbon dioxide and feeding at least a portion of the gas mixture to the hydrogenation step without separating hydrogen, carbon monoxide, and carbon dioxide.

For features and advantages of such a system, which is configured in particular for carrying out a method as explained above, reference is expressly made to the above explanations.

Embodiments are explained in more detail below with reference to two examples and then with reference to a drawing, which also illustrates an embodiment.

### Exemplary Embodiment I—Propylene Production

The following statements assume idealized conditions and reactions in the reforming and hydrogenation reactor or in corresponding method steps carried out according to embodiments (hereinafter, the terms “hydrogenation step” and “hydrogenation reactor” on the one hand and “reforming step” or “reforming reactor” on the other hand are used synonymously).

Here, a stream of carbon dioxide is fed to the process and is converted in the hydrogenation step into propylene according to Equation I.

3CO₂+9H₂→C₃H₆+6H₂O (I)

The fact that, in reality, only a partial conversion may occur here, does not play a role in the idealized model since unconverted carbon dioxide is returned into the hydrogenation via a corresponding separation, for example an amine scrubbing. The formation of other by-products also remains unconsidered. In particular for the formation of paraffins (here primarily propane), a comparable

computing model, in which a further mole of hydrogen per target molecule is then required, can be established. The overall process thus applies as the balance limit.

Furthermore, water and methane, for example as natural gas, are fed to the reforming step as input streams.

The following essential reactions occur in the reformer reactor: According to Equation II, one mole of carbon monoxide and three moles of hydrogen are formed from methane and water in each case. The water gas shift reaction according to Equation III as a side reaction can be left out of consideration since these products within the meaning of the disclosed embodiments are converted according to Equation I in the same stoichiometry in the hydrogenation step.

H₂O+CH₄→CO+3H₂ (II)

CO+H₂O→H₂+CO₂ (III)

Within the framework of embodiments, the carbon monoxide is advantageously also converted with hydrogen in the hydrogenation reactor into propylene according to Equation IV.

3CO+6H₂→C₃H₆+3H₂O (IV)

After passage through the hydrogenation reactor with conversion of carbon monoxide and hydrogen according to Equation IV, an excess of hydrogen, as results from the combination of Equations II and IV (Equation V), thus remains from the synthesis gas formed in the reforming reactor (Equation II).

3H₂O+3CH₄→3CO+9H₂→C₃H₆+3H₂O+3H₂ (V)

For covering the stoichiometric hydrogen demand of the propylene production according to Equation I, three times the molar quantities of water and methane must thus be converted in the reforming reactor. In one embodiment under real, non-idealized conditions, the ratio of synthesis gas to carbon dioxide can lie in the range of 6:1 to 2:1, in particular of 4:1 to 2.5:1.

In the idealized case, the following gross reaction Equation VI of the overall process can thus be established from Equations I, II, and IV:

3 CO₂+9 CH₄+9 H₂O→4 C₃H₆+15 H₂O (VI)

If the carbon dioxide stream used already contains portions of carbon monoxide and/or hydrogen, the hydrogen demand of the overall process and the quantity of input streams to the reforming reactor will be reduced accordingly.

In particular, hydrogen fractions in one of the input streams or else further separately fed hydrogen have a positive effect here.

### Exemplary Embodiment II—General Consideration

Building on Exemplary Embodiment I, consideration for olefins and paraffins of any chain length n is generalized in the following. The following applies to the hydrogenation reactor:

nCO₂+(3n+o)H₂→CₙH₂ₙ₊ₒ+2nH₂O (VII)

As mentioned, an olefin product is formed when o=0 and a paraffin product is formed when n=2. For consideration of the reforming reactor, only the aforementioned Equation II continues to be relevant as explained above.

Again, the carbon monoxide is advantageously also converted with hydrogen into the target product according to Equation VIII.

nCO+(2n+o)H₂→CₙH2ₙ₊ₒ+nH₂O (VIII)

After passage through the hydrogenation reactor with conversion of carbon monoxide and hydrogen according to Equation VIII, an excess of hydrogen, as results from the combination of Equations II and VIII (Equation IX), thus still remains from the synthesis gas formed in the reforming step (Equation II).

nH2O+nCH₄→nCO+3nH₂→CₙH2ₙ₊ₒ+nH₂O+(n−o)H₂ (IX)

In the idealized case, the following gross reaction Equation X of the overall process can thus again be established from Equations VII, II, and IX:

nCO₂+(3n+o)CH₄+(3n+o)H₂O→4CnH2ₙ₊ₒ+(5n+o)H₂O (X)

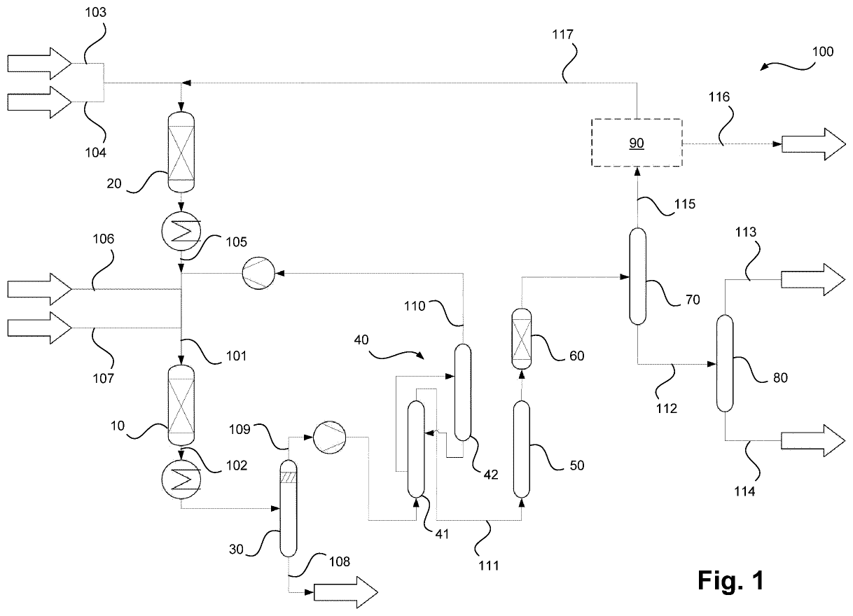

In the solitary FIG. 1, a method according to one embodiment of the disclosed embodiments is illustrated and denoted as a whole by 100.

In the method 100, which serves to produce propylene in the illustrated embodiment but generally serves to produce one or more olefins having a carbon number as described above, carbon dioxide and hydrogen are fed as input stream 101 to a hydrogenation step 10, wherein a portion of the carbon dioxide and a portion of the hydrogen that are fed to the hydrogenation step 10 in the input stream 101 are converted with one another in the hydrogenation step 10 on a bifunctional catalyst via an oxygenate as an intermediate into the one or more olefins. A product or process stream 102 is provided by means of the hydrogenation step.

The hydrogen that is fed to the hydrogenation step 10 in the input stream 101 is provided at least in part by means of a reforming step 20, in which methane fed in the form of an input stream 103 and water fed in the form of an input stream 104 are converted into carbon dioxide and hydrogen. The carbon dioxide fed to the hydrogenation step 10 in the input stream 101 is provided in part using the reforming step 20. It is contained, in addition to carbon monoxide and water, in a cooled product stream 105 of the reforming step 20, which is obtained by a cooling step not separately designated. As a result of the cooling of condensed water, the liquid phase can be discharged from the process. In another part, the carbon dioxide that is fed to the hydrogenation step 10 in the input stream 101 is provided independently of the reforming step 20, for which purpose a further input stream 106 is used. Further hydrogen can optionally be fed in the form of an input stream 107.

After a cooling, likewise not separately designated, the product stream 102 of the hydrogenation step is fed to a condensate separation 30 in which a water-containing condensate stream 108 is formed. The said condensate stream can be returned into the method 100 or discharged from the method 100. The product stream 102 freed from condensate is fed in the form of a process stream 109 to a compression (not separately designated) and thereafter, if needed, subjected to a carbon dioxide separation, for example comprising an amine scrubbing 41 with regeneration 42. A carbon dioxide-rich stream 110 can in this way be returned into the hydrogenation step 10 after a compression not separately designated.

The material stream 111 which has already been largely freed of carbon dioxide can be subjected, if needed, to an alkaline scrubbing 50 for removing carbon dioxide residues and then to a drying 60. It is thereafter subjected to a separation 70 of compounds boiling more easily than propylene.

A bottom stream 112 formed in the separation 70 is fed to a further separation 80, in which a propylene-rich stream 113 and a propane-rich stream 114, which, where applicable, also contains heavier components, are formed. Atop stream 115 of the separation 70, which stream contains compounds boiling more easily than propylene, for example unconverted carbon monoxide and unconverted hydrogen, methane, and, where applicable, ethylene, is optionally fed to an ethylene separation 90, which can in particular be carried out as an adsorptive separation and in which an ethylene stream 116 is formed. Remaining residue, or in the absence of ethylene separation 90, the entire top stream 115 is returned into the reforming step 20 in the form of a return stream 117.

1. A method for producing one or more olefins having a carbon number of two to eight, comprising:
feeding carbon dioxide and hydrogen to a hydrogenation step, wherein at least a portion of the carbon dioxide and at least a portion of the hydrogen that are fed to the hydrogenation step are converted with one another in the hydrogenation step on a bifunctional catalyst via an oxygenate as an intermediate into the one or more olefins, providing the hydrogen that is fed to the hydrogenation step at least in part by means of a reforming step in which methane and water are converted into hydrogen, carbon monoxide, and carbon dioxide, and in that the carbon dioxide that is fed to the hydrogenation step is provided in part using the reforming step and in part independently of the reforming step, wherein providing at least a portion of the hydrogen and a portion of the carbon dioxide by means of the reforming step comprises providing, using the reforming step, a gas mixture containing hydrogen, carbon monoxide, and carbon dioxide and feeding at least a portion of the gas mixture to the hydrogenation step without separating hydrogen, carbon monoxide, and carbon dioxide, wherein the carbon dioxide that is provided using the reforming step is fed to the hydrogenation step in a first gas stream, and wherein the carbon dioxide that is provided independently of the reforming step is fed to the hydrogenation step in a second gas stream.

2. The method according to claim 1, in which the methane is at least in part provided using natural gas.

3. The method according to claim 1, in which carbon monoxide is formed in the reforming step and fed to the hydrogenation step, wherein a molar ratio between carbon monoxide and carbon dioxide in a synthesis gas formed in in a in the reforming step is not less than 1:1.

4. The method according to claim 1, wherein a molar ratio between the carbon monoxide and carbon dioxide that are fed to the hydrogenation step is not more than 1:1 as a result of adding the carbon dioxide provided independently of the reforming step.

5. The method according to claim 1, in which a gas mixture is formed in the reforming step, and at least a portion of said gas mixture is fed to the hydrogenation step in a material composition that is unchanged except for the water content.

6. The method according to claim 1, in which a molar ratio between the carbon dioxide and the hydrogen that are fed to the hydrogenation step (10) is 1:6 to 1:1.

7. The method according to claim 1, in which a molar ratio between the methane and the water that are fed to the reforming step is 1:8 to 4:1.

8. The method according to claim 1, in which a quantity ratio between the first and the second gas stream is selected such that the components provided with the first gas stream and the second gas stream correspond to a stoichiometric demand in the hydrogenation step.

9. The method according to claim 1, in which the one or more olefins includes is propylene.

10. The method according to claim 1, in which at least a portion of the carbon dioxide that is fed to the hydrogenation step and/or at least a portion of the hydrogen that is fed to the hydrogenation step is not converted in the hydrogenation step.

11. The method (100) according to claim 1, in which the one or more olefins is removed from the hydrogenation step as part of a product mixture which, in addition to the one or more olefins, comprises at least carbon dioxide and/or hydrogen and/or one or more paraffins as further components, wherein at least a portion of the further components is separated out from the product mixture and returned in each case at least in part into the hydrogenation step and/or the reforming step.

12. The method according to claim 1 comprising:
providing a pressure of 10 to 100 bar in the reforming step and a pressure of 1 to 100 bar in the hydrogenation step at temperatures between 100 to 520° C.

13. The method according to claim 1, in which the reforming step is carried out at a higher pressure level than the hydrogenation step.

14. A system (100) for producing one or more olefins having a carbon number of two to eight, comprising:
a hydrogenation reactor configured to subject carbon dioxide and hydrogen to a hydrogenation step, in which a portion of the carbon dioxide and a portion of the hydrogen that are subjected to the hydrogenation step are converted with one another on a bifunctional catalyst via an oxygenate as an intermediate into the one or more olefins; and
a reforming reactor is configured to provide the hydrogen that is fed to the hydrogenation step and execute a reforming step, in which methane and water are converted into hydrogen, carbon monoxide, and carbon dioxide;
the system being configured to provide the carbon dioxide that is fed to the hydrogenation step, in part using the reforming step and in part independently of the reforming step, wherein providing at least a portion of the hydrogen, a portion of the carbon monoxide, and a portion of the carbon dioxide by means of the reforming step comprises providing, using the reforming step, a gas mixture containing hydrogen, carbon monoxide, and carbon dioxide and feeding at least a portion of the gas mixture to the hydrogenation step without separating hydrogen, carbon monoxide, and carbon dioxide; and
the system being further configured to feed the carbon dioxide that is provided using the reforming step, to the hydrogenation step in a first gas stream and to feed the carbon dioxide that is provided independently of the reforming step, to the hydrogenation step in a second gas stream.

15. (canceled)
